(19) United States
(12) Patent Application Publication
(10) Pub. No.: US 2023/0181749 A1
(43) Pub. Date: Jun. 15, 2023

(54) BICYCLIC PEPTIDE LIGANDS SPECIFIC FOR NECTIN-4 AND USES THEREOF

(71) Applicant: Bicycle Tx Limited, Cambridge (GB)

(72) Inventors: Amy Katherine DICKSON, Woburn, MA (US); Darren LIMB, Oxford (GB); Lisa MAHNKE, Boston, MA (US); Michael RIGBY, Cambridge (GB); Terrence Allen WEST, Boston, MA (US); David WITTY, Hertfordshire (GB)

(21) Appl. No.: 17/926,454

(22) PCT Filed: May 20, 2021
(86) PCT No.: PCT/GB2021/051220

§ 371 Date: Nov. 18, 2022

Related U.S. Application Data
(60) Provisional application No. 63/027,536, filed on May 20, 2020.

(51) Int. Cl. A61K 47/64; A61K 47/26; A61K 9/19

(52) U.S. Cl. CPC A61K 47/64; A61K 47/26; A61K 9/19

(57) ABSTRACT

The present invention relates to a Bicycle toxin conjugate BT8009, or pharmaceutically acceptable salts thereof, or pharmaceutical compositions thereof, and uses thereof. In one embodiment, the pharmaceutical composition comprises BT8009, histidine, sucrose and Polysorbate 20. In another embodiment, the pharmaceutical composition is for use in treating an advanced solid tumor malignancy associated with Nectin-4-expression in a patient, preferably in combination with Nivolumab.

### TECHNICAL FIELD OF THE INVENTION

The present invention relates to Bicycle toxin conjugates, or pharmaceutically acceptable salts thereof, or pharmaceutical compositions thereof. The present invention also provides uses of Bicycle toxin conjugates, or pharmaceutically acceptable salts thereof, or pharmaceutical compositions thereof, for preventing or treating a disease, disorder, or condition characterized by overexpression of Nectin-4 in diseased tissue.

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu et al. (2007), Science 330, 1066-71), a cyclic peptide with the Arg-Gly-Asp motif binding to integrin αVb3 (355 Å²) (Xiong et al. (2002), Science 296 (5565), 151-5) or the cyclic peptide inhibitor upain-1 binding to urokinase-type plasminogen activator (603 Å²; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8, (NIP-8) which lost its selectivity over other IMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example TATA (1,1′,1″-(1,3,5-triazinane-1,3,5-triyl)triprop-2-en-1-one, Heinis et al. Angew Chem, Int Ed. 2014; 53:1602-1606).

Phage display-based combinatorial approaches have been developed to generate and screen large libraries of bicyclic peptides to targets of interest (Heinis et al. (2009), Nat Chem Biol 5 (7), 502-7 and WO 2009/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule scaffold.

### SUMMARY OF THE INVENTION

According to one aspect, the invention provides a pharmaceutical composition comprising BT8009, or a pharmaceutically acceptable salt thereof, histidine, sucrose, and Polysorbate 20. In some embodiments, a pharmaceutical composition comprising BT8009, or a pharmaceutically acceptable salt thereof, histidine, sucrose, and Polysorbate 20 is a lyophilized powder. In some embodiments, a pharmaceutical composition comprising BT8009, or a pharmaceutically acceptable salt thereof, histidine, sucrose, and Polysorbate 20 is a pharmaceutical formulation in water.

In another aspect, the invention provides a method for treating advanced malignancies associated with Nectin-4 expression in a patient comprising administering to the patient a pharmaceutical composition as described herein. In some embodiments, the invention provides a method for treating advanced malignancies associated with Nectin-4 expression in a patient comprising administering to the patient weekly by IV infusion a pharmaceutical formulation comprising BT8009, or a pharmaceutically acceptable salt thereof, histidine, sucrose, and Polysorbate 20 in water.

### DETAILED DESCRIPTION OF CERTAIN EMBODIMENTS

### 1. General Description of Certain Embodiments of the Invention

In another aspect, the invention provides methods of using a pharmaceutical composition described herein for treating an advanced solid tumor malignancy associated with Nectin-4-expression.

In some embodiments, the invention provides a method for treating advanced malignancies associated with Nectin-4 expression in a patient comprising administering to the patient weekly by IV infusion over 1 hour a pharmaceutical formulation comprising BT8009, or a pharmaceutically acceptable salt thereof, histidine, sucrose, and Polysorbate 20 in water. In some embodiments, the pharmaceutical formulation comprising BT8009, or a pharmaceutically acceptable salt thereof, histidine, sucrose, and Polysorbate 20 in water is given as a single agent. In some embodiments, the pharmaceutical formulation comprising BT8009, or a pharmaceutically acceptable salt thereof, histidine, sucrose, and Polysorbate 20 in water is given in combination with nivolumab. In some embodiments, nivolumab is administered 240 mg over 30 minutes every 2 weeks. In some embodiments, the pharmaceutical formulation comprising BT8009, or a pharmaceutically acceptable salt thereof, histidine, sucrose, and Polysorbate 20 in water given in combination with nivolumab is administered by sequential infusions first with BT8009 followed by nivolumab.

In some embodiments, the advanced solid tumor malignancy associated with Nectin-4-expression is selected from the group consisting of non-small-cell lung cancer (NSCLC), ovarian cancer, triple-negative breast cancer (TNBC), gastric/upper gastrointestinal (GI) cancer, pancreatic cancer, and urothelial cancer.

In some embodiments, the advanced solid tumor malignancy associated with Nectin-4-expression is non-small-cell lung cancer (NSCLC). In some embodiments, the advanced solid tumor malignancy associated with Nectin-4-expression is ovarian cancer. In some embodiments, the advanced solid tumor malignancy associated with Nectin-4-expression is triple-negative breast cancer (TNBC). In some embodiments, the advanced solid tumor malignancy associated with Nectin-4-expression is gastric/upper gastrointestinal (GI). In some embodiments, the advanced solid tumor malignancy associated with Nectin-4-expression is pancreatic cancer. In some embodiments, the advanced solid tumor malignancy associated with Nectin-4-expression is urothelial cancer.

### 2. Compounds and Definitions

The term “BT8009,” as used herein, is a Bicycle toxin conjugate having a structure as shown below, wherein the molecular scaffold is 1,1′,1″-(1,3,5-triazinane-1,3,5-triyl)triprop-2-en-1-one (TATA), and the peptide ligand comprises the amino acid sequence:

(β-Ala)-Sar₁₀-CᵢP[1Nal][dD]CᵢᵢM[HArg]DWSTP[HyP]WCᵢᵢᵢ (SEQ ID NO: 1)

wherein Sar is sarcosine, 1Nal represents 1-naphthylalanine, HArg represents homoarginine, HyP represents hydroxyproline and Cᵢ, Cᵢᵢ and Cᵢᵢᵢ represent first, second and third cysteine residues.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

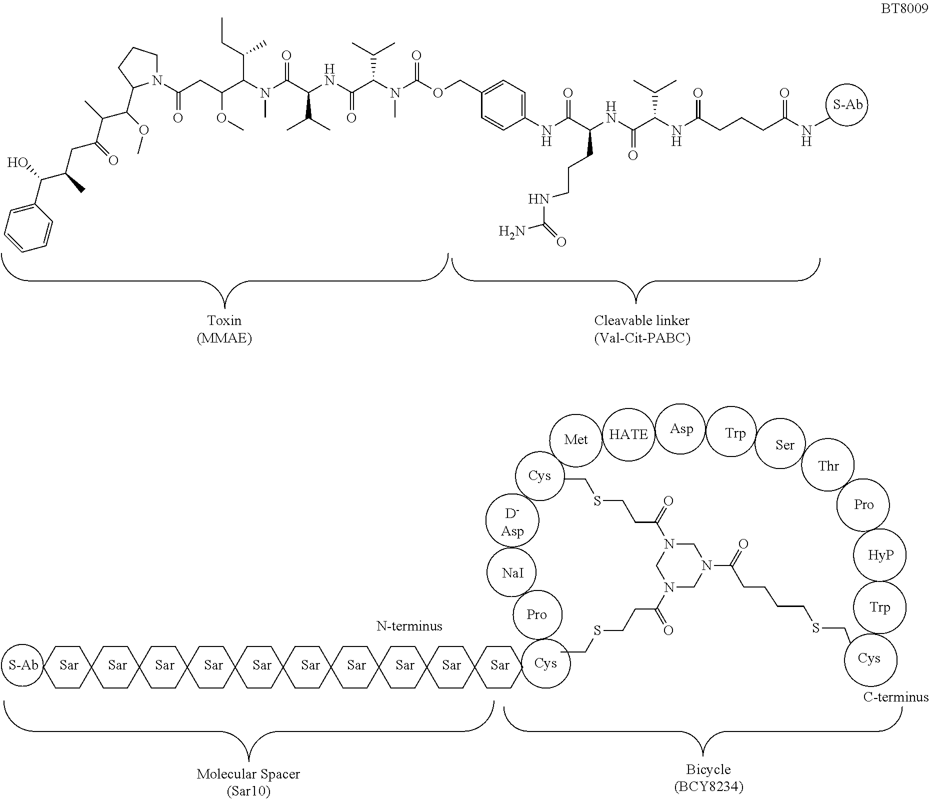

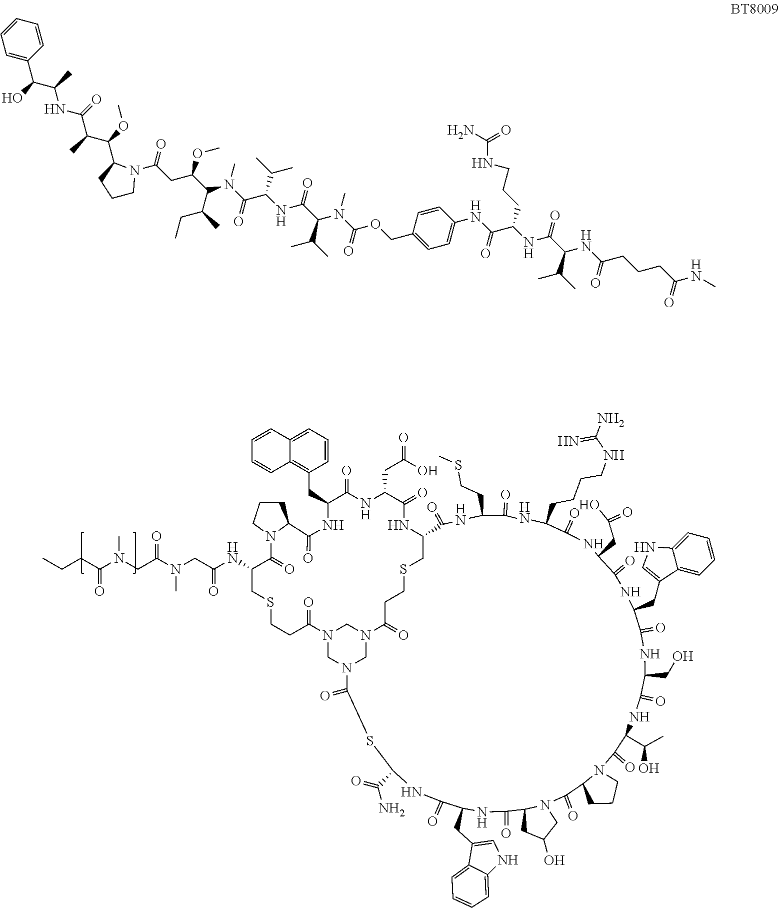

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

As used herein, the term “about” shall have the meaning of within 10% of a given value or range. In some embodiments, the term “about” refers to within 10%, 9%, 8%, 7%, 6%, 5%, 4%, 3%, 2%, or 1% of a given value.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

### 3. Pharmaceutical Compositions

According to one aspect, the invention provides a pharmaceutical composition comprising BT8009, or a pharmaceutically acceptable salt thereof, and a pharmaceutically acceptable excipient or carrier. In some embodiments, a pharmaceutical composition of the invention comprises about 21.2 mg BT8009, or a pharmaceutically acceptable salt thereof.

In some embodiments, a pharmaceutical composition of the invention is a solid pharmaceutical composition. In some embodiments, a solid pharmaceutical composition of the invention is powders. In some embodiments, a pharmaceutical composition of the invention is lyophilized powder. In some embodiments, a solid pharmaceutical composition of the invention is granules.

According to one aspect, the invention provides BT8009, or a pharmaceutically acceptable salt thereof, as a sterile lyophilized powder for solution. In some embodiments, the BT8009, or a pharmaceutically acceptable salt thereof, is contained in a 10 mL Type I clear glass vial with a chlorobutyl stopper and aluminum seal. In some embodiments, each vial includes 21.2 mg/vial of BT8009, or a pharmaceutically acceptable salt thereof, for reconstitution with 5.0 mL of water for injection (WFI). In some embodiments, a 4 mg/mL BT8009 solution is generated (the reconstituted drug substance contains histidine, sucrose, and Polysorbate 20), and 5.0 mL of the reconstituted solution is withdrawn to provide a 20 mg dose for further dilution with 0.9% saline and administration via IV infusion.

In some embodiments, a pharmaceutical composition of the invention is a liquid pharmaceutical composition. In some embodiments, a liquid pharmaceutical composition of the invention is a pharmaceutical formulation in an acceptable vehicle or solvent. In some embodiments, an acceptable vehicle or solvent is selected from sterile water, Ringer's solution, U.S.P. and isotonic sodium chloride solution. In some embodiments, an acceptable vehicle or solvent is sterile water. In some embodiments, an acceptable vehicle or solvent is a sterile injectable medium. In some embodiments, a liquid pharmaceutical composition of the invention comprises about 2-4 mg/mL BT8009, or a pharmaceutically acceptable salt thereof. In some embodiments, a liquid pharmaceutical composition of the invention comprises about 4 mg/mL BT8009, or a pharmaceutically acceptable salt thereof.

In some embodiments, a pharmaceutically acceptable excipient or carrier comprises a buffering agent. In some embodiments, a buffering agent is selected from phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. In some embodiments, a buffering agent is histidine. In some embodiments, a buffering agent is sodium hydroxide. In some embodiments, a buffering agent is hydrochloric acid.

In some embodiments, a buffering agent is at an amount to adjust pH of a pharmaceutical composition of the invention to about 6-8. In some embodiments, a buffering agent is histidine at an amount of about 1-3 mg per mg of BT8009, or a pharmaceutically acceptable thereof. In some embodiments, histidine is at an amount of about 1.31 or 2.62 mg per mg of BT8009, or a pharmaceutically acceptable thereof. In some embodiments, a liquid pharmaceutical composition of the invention comprises histidine at a concentration of about 5.24 mg/mL.

In some embodiments, a liquid pharmaceutical composition of the invention is at a pH of about 6-8. In some embodiments, a liquid pharmaceutical composition of the invention is at a pH of about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In some embodiments, a liquid pharmaceutical composition of the invention is at a pH of about 6.5 or 7.0.

In some embodiments, a pharmaceutically acceptable excipient or carrier comprises a stabilizer or cryoprotectant. In some embodiments, a stabilizer or cryoprotectant is dimethyl sulfoxide (DMSO). In some embodiments, a stabilizer or cryoprotectant is ethylene glycol. In some embodiments, a stabilizer or cryoprotectant is glycerol. In some embodiments, a stabilizer or cryoprotectant is propylene glycol. In some embodiments, a stabilizer or cryoprotectant is 2-methyl-2, 4-pentanediol (MPD). In some embodiments, a stabilizer or cryoprotectant is trehalose. In some embodiments, a stabilizer or cryoprotectant is formamide. In some embodiments, a stabilizer or cryoprotectant is proline. In some embodiments, a stabilizer or cryoprotectant is glycerol 3-phosphate. In some embodiments, a stabilizer or cryoprotectant is sorbitol. In some embodiments, a stabilizer or cryoprotectant is diethyl glycol. In some embodiments, a stabilizer or cryoprotectant is sucrose.

In some embodiments, a stabilizer or cryoprotectant (e.g., sucrose) is at an amount of about 10-35 mg per mg of BT8009, or a pharmaceutically acceptable thereof. In some embodiments, a stabilizer or cryoprotectant (e.g., sucrose) is at an amount of about 15 or 30 mg per mg of BT8009, or a pharmaceutically acceptable thereof. In some embodiments, a liquid pharmaceutical composition comprises a stabilizer or cryoprotectant (e.g., sucrose) at a concentration of about 60 mg/mL.

In some embodiments, a pharmaceutically acceptable excipient or carrier comprises a surfactant. In some embodiments, a surfactant is a polysorbate (e.g., polysorbate-20, polysorbate-40, polysorbate-60, polysorbate-65, polysorbate-80, polysorbate-85, or a combination thereof). In some embodiments, a surfactant is selected from poloxamers (e.g., poloxamer 188); Triton™; sodium dodecyl sulfate (SDS); sodium laurel sulfate; sodium octyl glycoside; lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauroamidopropyl-betaine, cocamidopropyl-betaine, linoleamidopropyl-betaine, myristamidopropyl-betaine, palmidopropyl-betaine, isostearamidopropyl-betaine (e.g. lauroamidopropyl), myristarnidopropyl-, palmidopropyl-, or isostearamidopropyl-dimethylamine; sodium methyl cocoyl-, or disodium methyl ofeyl-taurate; and the Monaquat™ series (Mona Industries, Inc., Paterson, N.J.), poly ethyl glycol, polypropyl glycol, and copolymers of ethylene and propylene glycol (e.g. pluronics, PF68). In some embodiments, a surfactant is Polysorbate 20.

In some embodiments, a surfactant (e.g., Polysorbate 20) is at an amount of about 0.01-0.15 mg per mg of BT8009, or a pharmaceutically acceptable thereof. In some embodiments, a surfactant (e.g., Polysorbate 20) is at an amount of about 0.025, 0.05, or 0.1 mg per mg of BT8009, or a pharmaceutically acceptable thereof. In some embodiments, a liquid pharmaceutical composition comprises a surfactant (e.g., Polysorbate 20) at a concentration of about 0.1 or 0.2 mg/mL.

In some embodiments, a pharmaceutically acceptable excipient or carrier comprises an isotonicity adjusting agent. In some embodiments, an isotonicity adjusting agent is sodium chloride, dextrose, calcium chloride, or a combination thereof. In some embodiments, an isotonicity adjusting agent is dextrose. In some embodiments, an isotonicity adjusting agent is sodium chloride. In some embodiments, an isotonicity adjusting agent is a combination of sodium chloride and dextrose.

In some embodiments, the invention provides a pharmaceutical composition comprising BT8009, or a pharmaceutically acceptable salt thereof, histidine, sucrose, and Polysorbate 20. In some embodiments, a pharmaceutical composition of the invention comprises:
BT8009, or a pharmaceutically acceptable salt thereof;
about 1.31-2.62 mg histidine per mg of BT8009, or a pharmaceutically acceptable thereof;
about 15-30 mg sucrose per mg of BT8009, or a pharmaceutically acceptable thereof; and
about 0.05-0.1 mg Polysorbate 20 per mg of BT8009, or a pharmaceutically acceptable thereof.

In some embodiments, the invention provides a solid pharmaceutical composition, which is a lyophilized powder, comprising:
about 21.2 mg BT8009, or a pharmaceutically acceptable salt thereof;
about 27.8 mg histidine;
about 318 mg sucrose; and
about 1.06 mg Polysorbate 20.

In some embodiments, the invention provides a liquid pharmaceutical composition comprising:
about 2-4 mg/mL BT8009, or a pharmaceutically acceptable salt thereof;
about 5.24 mg/mL histidine;
about 60 mg/mL sucrose; and
about 0.2 mg/mL Polysorbate 20.

In some embodiments, the invention provides a liquid pharmaceutical composition prepared by dissolving a solid pharmaceutical composition of the invention in water. In some embodiments, the invention provides a liquid pharmaceutical composition prepared by dissolving a solid pharmaceutical composition of the invention in an injectable medium (e.g., 0.9% w/v saline or 5% dextrose). In some embodiments, the invention provides a liquid pharmaceutical composition prepared by reconstituting a solid pharmaceutical composition of the invention in water, followed by dilution with 0.9% w/v saline. In some embodiments, a liquid pharmaceutical composition is diluted into a 0.9% w/v saline IV bag for IV administration.

In some embodiments, the invention provides a solid pharmaceutical composition comprising BT8009, or a pharmaceutically acceptable salt thereof, histidine, sucrose, and Polysorbate 20. In some embodiments, the invention provides a liquid pharmaceutical composition comprising BT8009, or a pharmaceutically acceptable salt thereof, histidine, sucrose, Polysorbate 20, and water. In some embodiments, the components of the pharmaceutical compositions are at the amount, concentration, and ratio as described above.

### 4. Uses of the Pharmaceutical Compositions

In one aspect, the invention provides a method, or a use, for treating an advanced solid tumor malignancy associated with Nectin-4-expression in a patient comprising administering to the patient a pharmaceutical composition as described herein. In some embodiments, an advanced solid tumor malignancy associated with Nectin-4-expression is selected from non-small-cell lung cancer (NSCLC), ovarian cancer, triple-negative breast cancer (TNBC), gastric/upper gastrointestinal (GI), pancreatic and urothelial cancers. In some embodiments, an advanced solid tumor malignancy associated with Nectin-4-expression is an adenocarcinoma subtype of NSCLC (adeno-NSCLC).

In some embodiments, a method of the invention comprises administering to a patient intravenously a pharmaceutical composition as described herein. In some embodiments, a pharmaceutical composition of the invention is administered by an IV injection. In some embodiments, a pharmaceutical composition of the invention is administered by an IV infusion. In some embodiments, an IV infusion of a pharmaceutical composition of the invention lasts about 5-30 minutes. In some embodiments, an IV infusion of a pharmaceutical composition of the invention lasts about 30-90 minutes. In some embodiments, an IV infusion of a pharmaceutical composition of the invention lasts about 30, 35, 40, 45, 50, 55, 60, 65, 70, 75, 80, 85, or 90 minutes. In some embodiments, an IV infusion of a pharmaceutical composition of the invention lasts about 60 minutes. In some embodiments, an IV infusion of a pharmaceutical composition of the invention lasts about 2, 2.5, 3, 3.5, or 4 hours.

In some embodiments, a pharmaceutical composition of the invention is administered to a patient once every 1, 2, 3, 4, 5, 6, or 7 days. In some embodiments, a pharmaceutical composition of the invention is administered to a patient weekly. In some embodiments, a pharmaceutical composition of the invention is administered to a patient once every two weeks.

In some embodiments, a pharmaceutical composition of the invention is administered at a dose of about 1-27 mg/m². In some embodiments, a pharmaceutical composition of the invention is administered at a dose of about 2-20 mg/m². In some embodiments, a pharmaceutical composition of the invention is administered at a dose of about 2-20 mg/m². In some embodiments, a pharmaceutical composition of the invention is administered at a dose of about 2.2, 4.4, 7.3, 11, 14.6, or 19.4 mg/m². In some embodiments, a pharmaceutical composition of the invention is administered at a dose of about 2.5, 5.0, 7.5, 10.0, 13.0, or 17.0 mg/m². In some embodiments, a pharmaceutical composition of the invention is administered at a dose of about 1.5-3.5, 3.5-5.5, 6.5-8.5, 10-12, 13.5-15.5, or 18.5-20.5 mg/m². In some embodiments, a pharmaceutical composition of the invention is administered at a dose of about 1-10 or 10-20 mg/m². In some embodiments, a pharmaceutical composition of the invention is administered at a dose of about 21, 22, 23, 24, 25, 26, or 27 mg/m².

In some embodiments, a pharmaceutical composition of the invention is administered to a patient at least 18 years-of-age.

In some embodiments, a pharmaceutical composition of the invention is administered to a patient having an Eastern Cooperative Oncology Group (ECOG) Performance Status score of 0 or 1. The ECOG Performance Status scores of 0 and 1 are described in Example 1.

In some embodiments, a pharmaceutical composition of the invention is administered to a patient having measurable disease per Response Evaluation Criteria in Solid Tumors (RECIST) v1.1.

In some embodiments, a pharmaceutical composition of the invention is administered to a patient having acceptable organ function. In some embodiments, a patient having acceptable organ function has laboratory data selected from the following:
Renal function: creatinine clearance of ≥50 mL/min by the Cockcroft-Gault equation or as measured by 24-hour urine collection;
Total bilirubin ≤1.5×ULN (upper limit of normal);
Serum albumin ≥2.5 g/dL;
Aspartate aminotransferase (AST) ≤2.5×ULN or ≤5×ULN in the presence of liver metastases;
Alanine aminotransferase (ALT)≤2.5×ULN or ≤5×ULN in the presence of liver metastases; and
International normal ratio (INR) <1.3 or ≤institutional ULN (anticoagulants not allowed).

In some embodiments, a pharmaceutical composition of the invention is administered to a patient having acceptable hematologic function. In some embodiments, a patient having acceptable hematologic function has laboratory data selected from the following:
Hemoglobin ≥9 g/dL;
Absolute neutrophil count (ANC) ≥1500 cells/mm³; and
Platelet count ≥75,000 cells/mm³.

In some embodiments, a pharmaceutical composition of the invention is administered to a patient who is a women of childbearing potential (WOCBP) that has a negative pregnancy test (negative serum test at screening and negative urine or serum test within 3 days prior to the first dose of a pharmaceutical composition of the invention)

In some embodiments, a pharmaceutical composition of the invention is administered to a patient who has a life expectancy ≥12 weeks after the start of BT8009 treatment according to the Investigator judgment.

In some embodiments, a pharmaceutical composition of the invention is administered to a patient with an advanced, histologically confirmed malignant solid tumor as follows: a) urothelial (transitional cell) carcinoma naïve to Nectin-4 directed therapies; or b) having tumor tissue (fresh biopsy or on archived sample less than 12 months old without intervening anti-cancer therapies) testing positive for Nectin-4 expression; or c) solid tumors known to be historically associated with Nectin-4 as follows: pancreatic, TNBC, NSCLC, gastric, esophageal or ovarian.

In some embodiments, a pharmaceutical composition of the invention is administered to a patient with disease that recurred after or been refractory to previous therapy including appropriate targeted therapies, for example EGFR or ALK therapies for relevant oncogene driver NSCLC patients, and are candidates for a Phase I study due to lack of approved or standard options for treatment.

In some embodiments, a pharmaceutical composition of the invention is administered to a patient with solid tumor metastatic recurrent disease confirmed to express Nectin-4 on fresh biopsy or archived tissue (less than 12 months old and with no intervening anti-cancer therapies) are eligible and must have exhausted all standard treatment options, including appropriate targeted therapies, for example EGFR or ALK therapies for relevant oncogene driver NSCLC patients, must have failed at least one prior line of therapy with evidence of radiographic progression on the most recent line of therapy.

In some embodiments, a pharmaceutical composition of the invention is administered to a patient naïve to Nectin-4 directed therapies.

In some embodiments, a pharmaceutical composition of the invention is administered to a patient with a solid tumor advanced disease having exhausted all appropriate standards of care options as follows: 1) patient with GFR (by CG or by 24-hour urine) 40-50 ml/min (or equivalent units) to be enrolled and evaluated first (refer to SRC section) followed by; 2) a patient with GFR 30-40 ml/min (or equivalent units).

In some embodiments, a patient has not had a chemotherapy treatment within 14 days prior to first dose of a pharmaceutical composition of the invention. In some embodiments, a patient has not had an anticancer treatment within 28 days or 5 half-lives, whichever shorter, prior to first dose of a pharmaceutical composition of the invention. In some embodiments, a patient has prior toxicities which have resolved to grade 1 per Common Terminology Criteria for Adverse Events (CTCAE) v 5.0 (except alopecia which must be no greater than Grade 2).

In some embodiments, a patient has not had an experimental treatment within 4 weeks of first dose of a pharmaceutical composition of the invention.

In some embodiments, a patient has not had prior treatment with Nectin-4 targeted therapy.

In some embodiments, a patient does not have a current treatment with strong inhibitors or inducers of CYP3A4 or strong inhibitors of P-gp including herbal- or food-based.

In some embodiments, a patient does not have any sensitivity to any of the ingredients of a pharmaceutical composition of the invention, or to monomethyl auristatin E (MMAE).

In some embodiments, a patient does not have a weight >100 kg (222.2 lbs) or BSA >2.21 m² (representing human 100 kg equivalent for height 175 cm).

In some embodiments, a patient does not have a significant medical condition including but not limited eye (conditions related to or that may confound monitoring for dry eye, corneal opacities or keratitis); skin (conditions related to or that may confound monitoring for rash including but not limited to autoimmune conditions such as eczema or psoriasis), life-threatening illness, active uncontrolled infection or organ system dysfunction (such as ascites, coagulopathy, encephalopathy), or other reasons which could compromise the patient's safety, or interfere with or compromise the integrity of the study outcomes including consideration of gastrointestinal, skin and pulmonary co-morbidities and including review of screening chest CT to ensure no clinically significant co-morbidities.

In some embodiments, a patient has a prior ≤Grade 2 thyroid endocrinopathy, if appropriately controlled with thyroid hormone and stable for at least 2 months on therapy.

In some embodiments, a patient does not have a clinically relevant troponin elevation.

In some embodiments, a patient does not have uncontrolled diabetes defined as hemoglobin A1C (HbA1c) ≥8%.

In some embodiments, a patient has not had major surgery (excluding placement of vascular access) within 4 weeks of first dose of a pharmaceutical composition of the invention and has recovered adequately prior to starting study therapy.

In some embodiments, a patient has not received a live vaccine within 30 days of study treatment.

In some embodiments, a patient does not have uncontrolled, symptomatic brain metastases (must have stable neurologic status following local therapy for at least 4 weeks without the use of steroids or on stable or decreasing dose of less than or equal to 10 mg daily prednisone or equivalent at time of study treatment initiation and must be without neurologic dysfunction that would confound the evaluation of neurologic and other AEs).

In some embodiments, a patient does not have uncontrolled hypertension (systolic blood pressure [BP] Systolic BP >=160 mm Hg or diastolic BP >=100 mm Hg) prior to first dose of BT8009 (must have been in stable control for at least 2 months).

In some embodiments, a patient does not have a history or current evidence of any condition, therapy or laboratory abnormality that might confound the results of the study, interfere with the patient's participation, or is not in the best interest of the patient to participate in the opinion of the Investigator.

In some embodiments, a patient does not have a history of a cerebral vascular event (stroke or transient ischemic attack), unstable angina, myocardial infarction, congestive heart failure or symptoms of New York Heart Association Class III-IV documented within 6 months prior to first dose of BT8009 or: a. mean resting corrected QT interval (QTcF) >470 msec, b. any factors that increase the risk of QTc prolongation or risk of arrhythmic events such as heart failure, hypokalemia, congenital long QT syndrome, family history of long QT syndrome or unexplained sudden death under 40 years-of-age, or any concomitant medication known to prolong the QT interval, or c. any clinically important abnormalities (as assessed by the Investigator) in rhythm, conduction, or morphology of resting electrocardiograms (ECGs), e.g., complete left bundle branch block, third degree heart block.

In some embodiments, a patient does not have mean resting corrected QT interval (QTcF) >470 msec within 6 months prior to first dose of a pharmaceutical composition of the invention.

In some embodiments, a patient does not have, within 6 months prior to first dose of a pharmaceutical composition of the invention, any factors that increase the risk of QTc prolongation or risk of arrhythmic events such as heart failure, hypokalemia, congenital long QT syndrome, family history of long QT syndrome or unexplained sudden death under 40 years-of-age, or any concomitant medication known to prolong the QT interval.

In some embodiments, a patient does not have, within 6 months prior to first dose of a pharmaceutical composition of the invention, any clinically important abnormalities in rhythm, conduction, or morphology of resting electrocardiograms (ECGs), e.g., complete left bundle branch block, third degree heart block.

In some embodiments, a patient does not have human immunodeficiency virus (HIV) or acquired immune deficiency syndrome (AIDS).

In some embodiments, a patient does not have a positive hepatitis B surface antigen and/or anti-hepatitis B core antibody.

In some embodiments, a patient has a negative polymerase chain reaction (PCR) assay and has an appropriate antiviral therapy.

In some embodiments, a patient has an active hepatitis C infection with positive viral load if hepatitis C virus (HCV) antibody positive.

In some embodiments, a patient has been treated for hepatitis C infection and has sustained virologic response of ≥12 weeks.

In some embodiments, a patient does not have another malignancy within 3 years before the first dose of a pharmaceutical composition of the invention. In some embodiments, a patient does not have any residual disease from a previously diagnosed malignancy (excluding adequately treated with curative intent basal cell carcinoma, squamous cell of the skin, cervical intraepithelial neoplasia/cervical carcinoma in situ or melanoma in situ or ductal carcinoma in situ of the breast).

In some embodiments, a patient does not have systemic IV anti-infective treatment or fever within the last 14 days prior to first dose of a pharmaceutical composition of the invention.

In some embodiments, a patient does not have a suspicion of relevant and recent systemic viral syndrome or need for quarantine/isolation that is not resolved in the opinion of the investigator.

In some embodiments, a patient does not have a psychological, familial, sociological, or geographical conditions that does not permit compliance with the protocol and/or follow-up procedures.

In some embodiments, the invention provides a combination use of a pharmaceutical composition of the invention and Nivolumab, for treating an advanced solid tumor malignancy associated with Nectin-4-expression. Nivolumab can be administered as described on the label, which can be found at https://www.opdivohcp.com/dosing/dosing-schedules, the content of which is incorporated herein by reference in its entirety. In some embodiments, Nivolumab is administered 240 mg every 2 weeks. In some embodiments, Nivolumab is administered 480 mg every 4 weeks. In some embodiments, Nivolumab is administered as a 30-minute IV infusion.

In some embodiments of a combination use, a patient is not previously known being intolerance to an immune checkpoint inhibitor. In some embodiments of a combination use, a patient is not known being hypersensitivity to checkpoint inhibitor therapy. In some embodiments of a combination use, a patient has no prior organ transplant. In some embodiments of a combination use, a patient is not previously diagnosed with clinically relevant immunodeficiency. In some embodiments of a combination use, a patient does not have active systemic infection requiring therapy. In some embodiments of a combination use, a patient does not take more than 10 mg daily prednisone equivalent or other strong immunosuppressant. In some embodiments of a combination use, a patient does not have a history of autoimmune disease except alopecia or vitiligo. In some embodiments of a combination use, a patient does not have a history of interstitial lung disease.

### EXEMPLIFICATION

The following examples are intended to illustrate the invention and are not to be construed as being limitations thereon. All amino acids, unless noted otherwise, were used in the L-configurations.

### List of Abbreviations

| Abbreviation or | |
|---|---|
| special term | Explanation |
| ADA | Anti-drug antibody |
| ADC | Antibody-drug conjugate |
| AE | Adverse event |
| ALP | Alkaline phosphatase |
| ALT | Alanine aminotransferase |
| ANC | Absolute neutrophil count |
| AR | Adverse reaction |
| AST | Aspartate aminotransferase |
| BLRM | Bayesian Logistic Regression Model |
| BP | Blood pressure |
| BSA | Body surface area |
| BTC | Bicycle toxin conjugate |
| CFR | Code of Federal Regulations |
| CHMP | Committee for Medicinal Products for Human |
| | Use |
| CI | Confidence interval |
| Cₘₐₓ | Maximum plasma drug concentration |
| CR | Complete response |
| CRM | Continual reassessment method |
| CT | Computed tomography |
| ctDNA | Circulating tumor DNA |
| Development | Sarah Cannon Development |
| Innovations | Innovations |
| DLT | Dose-limiting toxicity |
| DOR | Duration of response |
| DRF | Dose-range finding |
| ECG | Electrocardiogram |
| ECOG | Eastern Cooperative Oncology Group |
| eCRF | Electronic Case Report Form |
| EGFR | Epidermal growth factor receptor |
| EMA | European Medicines Agency |
| EOI | End of infusion |
| EOT | End of treatment |
| EWOC | Escalation with overdose control |
| FAS | Full Analysis Set |
| Fc | Fragment crystallizable (region) |
| FDA | Food and Drug Administration, US |
| FFPE | Formalin-fixed paraffin-embedded |
| FIH | First in human |
| FSH | Follicle-stimulating hormone |
| GCP | Good Clinical Practice |
| GI | Gastrointestinal |
| GLP | Good Laboratory Practice |
| HED | Human equivalent dose |
| HIPPA | Health Insurance Portability and Accountability |
| | Act |
| HNSTD | Highest non-severely toxic dose |
| HR | Heart rate |
| HTN | Hypertension |
| IB | Investigator Brochure |
| ICF | Informed consent form |
| ICH | International Council for Harmonisation |
| IND | Investigational New Drug |
| INR | International normalized ratio |
| irAE | Immune-related adverse events |
| IRB | Institutional Review Board |
| ISF | Investigator Study File |
| IV | Intravenous |
| LDH | Lactate dehydrogenase |
| MAD | Maximum administrated dose |
| MMAE | Monomethyl auristatin E |
| MRI | Magnetic resonance imaging |
| MTD | Maximum tolerated dose |
| NCI CTCAE | National Cancer Institute Common |
| | Terminology Criteria for Adverse Events |
| NE | Not evaluable |
| NHP | Non-human primate |
| NSCLC | Non-small-cell lung cancer |
| OR | Objective response |
| ORR | Objective response rate |
| OS | Overall survival |
| PD | Progressive disease |
| PD-1 | Programmed cell death protein 1 |
| PD-L1 | Programmed death-ligand 1 |
| PDx | Pharmacodynamic |
| PE | Physical examination |
| PET | Positron emission tomography |
| PFS | Progression-free survival |
| PHI | Protected health information |
| PK | Pharmacokinetic |
| PR | Partial response |
| PT | Prothrombin time |
| PTT | Partial thromboplastin time |
| QT | ECG interval measured from the onset of the |
| | QRS complex to the end of the T wave |
| QTc | QT interval corrected for heart rate |
| REC | Research Ethics Committee |
| RECIST | Response Evaluation Criteria in Solid Tumors |
| RP2D | Recommended Phase II dose |
| SAE | Serious adverse event |
| SAP | Statistical Analysis Plan |
| SAR | Suspected adverse reaction |
| SAS | Safety Analysis Set |
| SD | Stable disease |
| SOA | Schedule of Assessments |
| SRC | Safety Review Committee |
| SUSAR | Suspected unexpected serious adverse reaction |
| TEAE | Treatment-emergent adverse event |
| TNBC | Triple negative breast cancer |
| UAE | Unexpected adverse event |
| ULN | Upper limit of normal |
| USPI | US Package Insert |
| WOCBP | Women of childbearing potential |

### Example 1. Phase I/II Study of the Safety, Pharmacokinetics, and Preliminary Clinical Activity of BT8009 in Patients with Advanced Malignancies Associated with Nectin-4 Expression

Number of Patients: Up to 146 patients are planned to be enrolled in this study; 34 in Part A-20 in Part A-2, 40 in each of the two Part B cohorts and 12 in Part C cohort.

Objectives: Primary Objectives

Primary Objectives

Number of Patients: Up to 146 patients are planned to be enrolled in this study; 34 in Part A-20 in Part A-2, 40 in each of the two Part B cohorts and 12 in Part C cohort.

Objectives: Primary Objectives

Primary Objectives

The primary objectives of the escalation and renal cohorts (Parts A-1, A-2, Part C) are:

To assess safety and tolerability of BT8009 in patients with advanced solid tumor malignancies associated with Nectin-4 expression as a monotherapy (Part A-1) or in combination with nivolumab (Part A-2) or in patients with advanced solid tumor malignancies having renal insufficiency (Part C).

To define the maximum tolerated dose (MTD) of BT8009, if observed, and determine a recommended Phase II dose (RP2D) as a monotherapy and in combination with nivolumab (Parts A-1 and A-2).

The primary objective of the expansion cohort (Parts B-1 and B-2) is:

To assess the clinical activity of BT8009 in patients with solid tumor indications associated with Nectin-4 expression as a monotherapy (Part B-1) and in combination with nivolumab (Part B-2) using RECIST 1.1

Secondary Objectives

The secondary objectives of the escalation (Parts A-1 and A-2) and the renal cohorts (Part C) are:

To assess preliminary signals of anti-tumor activity achieved with BT8009 administration in patients with advanced solid tumor malignancies associated with Nectin-4 as a monotherapy (Parts A-1) and in combination with nivolumab (Parts A-2) or in patients with advanced solid tumor malignancies having renal insufficiency (Part C).

To determine pharmacokinetic (PK) parameters of BT8009 in patients with advanced solid tumor malignancies associated with Nectin-4 as a monotherapy (Parts A-1) and in combination with nivolumab (Parts A-2) or in patients with advanced solid tumor malignancies having renal insufficiency (Part C).

To determine incidence of anti-drug antibody (ADA) development

The secondary objectives of the expansion cohorts (Parts B-1 and B-2) are:

To assess safety and tolerability of BT8009 in patients with solid tumor indications associated with Nectin-4 as a monotherapy (Part B-1) and in combination with nivolumab (Part B-2)

To determine pharmacokinetic (PK) parameters of BT8009

To determine incidence of anti-drug antibody (ADA) development

Study Design

This study is a Phase I/II, first-in-human, open-label dose-escalation study of BT8009 given as a single agent (Parts A-1, B-1 and C) and in combination with nivolumab (Parts A-2 and B-2). There are three parts to this study: Part A, dose escalation, Part B, dose expansion and Part C, renal insufficiency. The study schema with a representative potential scheme is presented in FIG. 3.

Study Drugs, Doses, and Modes of Administration:

BT8009, in ascending doses in dose escalation cohorts (A-1 and A-2) and at the RP2D in expansion cohorts (B-1 and B-2), and renal cohort (C) administered intravenously as infusion over 1 h.

Nivolumab administered 240 mg over 30 minutes every 2 weeks for combination therapy in Part A-2 and B-2.

Inclusion Criteria—All Patients:

Patients must meet the following criteria in order to be included in the research study:
1. Written informed consent, according to local guidelines, signed and dated by the patient or by a legal guardian prior to the performance of any study-specific procedures, sampling, or analyses.
If a patient declines to participate in any voluntary component of the study (e.g., tumor biopsy), there will be no penalty or loss of benefit to the patient, and he/she will not be excluded from other aspects of the study.
2. At least 18 years-of-age at the time of signature of the informed consent form
3. Eastern Cooperative Oncology Group (ECOG) Performance Status score of 0 or 1 (see Appendix A)
Patients must have measurable disease per Response Evaluation Criteria in Solid Tumors (RECIST) v1.1 (see Appendix B)
5. Acceptable organ function, as evidenced by the following laboratory data:
a. Renal function, as follows: creatinine clearance of ≥50 mL/min by the Cockcroft-Gault equation or equivalent.
b. Total bilirubin ≤1.5×ULN (upper limit of normal)
c. Serum albumin ≥2.5 g/dL
d. Aspartate aminotransferase (AST) ≤2.5×ULN or ≤5×ULN in the presence of liver metastases
e. Alanine aminotransferase (ALT) ≤2.5×ULN or ≤5×ULN in the presence of liver metastases
f. International normal ratio (INR) <1.3 or ≤institutional ULN
6. Acceptable hematologic function (no red blood cell or platelet transfusions or growth factors are allowed within 2 weeks of the first dose of BT8009):
a. Hemoglobin ≥9 g/dL
b. Absolute neutrophil count (ANC) ≥1500 cells/mm3
c. Platelet count ≥75,000 cells/mm3
7. Negative pregnancy test for women of childbearing potential (WOCBP) (negative serum test at screening and negative urine or serum test within 3 days prior to the first dose of BT8009. Definition of non-WOCBP is in Appendix C. Male patients with female partners of childbearing potential and female patients of childbearing potential are required to follow highly effective contraception (oral and hormonal contraceptives allowed) at least as conservative as Clinical Trial Facilitation Group (CTFG) recommendations for less than 1% failure rate (https://www.hma.eu/fileadmin/dateien/Human_Medicines/01-About_HMA/Working_Groups/CTFG/2014_09_HMA_CTFG_Contraception.pdf), (Appendix C) during their participation in the study and for 6 months following last dose of study drug. Male patients must also refrain from donating sperm during their participation in the study for 6 months following last dose of study drug and women must not breastfeed during that time or donate eggs.
8. Availability of archived tumor samples within 12 months prior to the date of the first dose of BT8009 or willingness to provide fresh tumor biopsy during screening.
9. Life expectancy ≥12 weeks after the start of BT8009 treatment according to the Investigator judgment.
10. Must be willing and able to comply with the protocol and study procedures.

### Additional Inclusion Criteria—Part A Only

11. Patients with advanced, histologically confirmed malignant solid tumors as follows:
a) urothelial (transitional cell) carcinoma naïve to Nectin-4 directed therapies; or
b) having tumor tissue (fresh biopsy or on archived sample less than 12 months old without intervening anti-cancer therapies) testing positive for Nectin-4 expression; or
c) solid tumors known to be historically associated with Nectin-4 as follows: pancreatic, TNBC, NSCLC, gastric, esophageal or ovarian.
Exceptions: single-subject accelerated cohorts may enroll patients with advanced solid tumors non-restricted to the above definitions, unless SRC views otherwise. All patients must have disease that recurred after or been refractory to previous therapy including appropriate targeted therapies, for example EGFR or ALK therapies for relevant oncogene driver NSCLC patients, and are candidates for a Phase I study due to lack of approved or standard options for treatment. The Sponsor may require a), b), or c) at any time during the enrollment. The Sponsor and/or SRC may decide to require enrollment of specific tumor (sub)types at any point during the escalation if it is felt necessary to enrich the evaluation of biomarkers, safety, anti-tumor activity, or PK.

Additional Inclusion Criteria—Part B-1 and B-2 Nectin-4 Basket Monotherapy and Combination Cohorts

12. Patients with solid tumor metastatic recurrent disease confirmed to express Nectin-4 on fresh biopsy or archived tissue (less than 12 months old and with no intervening anti-cancer therapies) are eligible and must have exhausted all standard treatment options, including appropriate targeted therapies, for example EGFR or ALK therapies for relevant oncogene driver NSCLC patients, must have failed at least one prior line of therapy with evidence of radiographic progression on the most recent line of therapy. Patients must be naïve to Nectin-4 directed therapies. Sponsor may decide not to allow urothelial (transitional cell) patients. If patient's tumor has been demonstrated to contain a therapeutically targetable somatic or driver mutation, therapy must be given with appropriate locally-approved therapy, then therapy must have been given based on local standard standards of care. If platinum therapy is applicable, then FDA-approved or appropriate locally-approved therapy must have been given based on local standard guidelines. If prior immunotherapy, the last dose must have been at least 28 days prior to the first dose of BT8009. The Sponsor and/or SRC may decide to require enrollment of specific tumor (sub)types meeting the above definition at any point during the expansion if it is felt necessary to enrich the population for anti-tumor activity.
13. At least 6 patients per cohort must have at least 1 tumor lesion amenable to biopsy and must be willing to undergo a biopsy prior to first dose of BT8009 and following any dose in Cycle 1.

Additional Inclusion Criteria—Part C Renal Insufficiency Cohort

14. Patients with solid tumor advanced disease having exhausted all appropriate standards of care options are eligible as follows: 1) 6 patients with GFR (by CG or by 24-hour urine) 40-50 ml/min (or equivalent units) to be enrolled and evaluated first (refer to SRC section) followed by; 2) 6 patients with GFR 30-40 ml/min (or equivalent units). Sponsor may opt to specify tumor (sub)type or Nectin-4 selected.

Exclusion Criteria—All Patients:

Patients who meet any of the following criteria will be excluded from study entry:
1. Chemotherapy treatments within 14 days prior to first dose of study treatment, other anticancer treatments, treatment within 28 days or 5 half-lives, whichever is shorter. Prior toxicities must have resolved to Grade 1 per Common Terminology Criteria for Adverse Events (CTCAE) v 5.0 (except alopecia which must be no greater than Grade 2).
2. Experimental treatments within 4 weeks of first dose of BT8009.
3. Prior treatment with Nectin-4 targeted therapy
4. Current treatment with strong inhibitors or strong inducers of CYP3A4 or strong inhibitors of P-gp including herbal- or food-based (Appendix F).
5. Known sensitivity to any of the ingredients of the investigational product or monomethyl auristatin E (MMAE).
6. Weight >100 kg (222.2 lbs) or BSA >2.21 m² (representing human 100 kg equivalent for height 175 cm).
7. Significant medical condition including but not limited eye (conditions related to or that may confound monitoring for dry eye, corneal opacities or keratitis); skin (conditions related to or that may confound monitoring for rash including but not limited to autoimmune conditions such as eczema or psoriasis), life-threatening illness, active uncontrolled infection or organ system dysfunction (such as ascites, coagulopathy, encephalopathy), or other reasons which, in the Investigator opinion, could compromise the patient's safety, or interfere with or compromise the integrity of the study outcomes including consideration of gastrointestinal, skin and pulmonary co-morbidities and including review of screening chest CT to ensure no clinically significant co-morbidities. Prior ≤Grade 2 thyroid endocrinopathy is allowed, if appropriately controlled with thyroid hormone and stable for at least 2 months on therapy.
8. Clinically relevant troponin elevation (considering local reference standards).
9. Uncontrolled diabetes defined as either a) hemoglobin A1C (HbA1c) ≥8%
10. Major surgery (excluding placement of vascular access) within 4 weeks of first dose of BT8009 and must have recovered adequately prior to starting study therapy
11. Receipt of live vaccine within 30 days of study treatment
12. Uncontrolled, symptomatic brain metastases (must have stable neurologic status following local therapy for at least 4 weeks without the use of steroids or on stable or decreasing dose of less than or equal to 10 mg daily prednisone or equivalent at time of study treatment initiation and must be without neurologic dysfunction that would confound the evaluation of neurologic and other AEs.)
13. Patients with uncontrolled hypertension (systolic blood pressure [BP] Systolic BP >=160 mm Hg or diastolic BP >=100 mm Hg) prior to first dose of BT8009 (must have been in stable control for at least 2 months)
14. History or current evidence of any condition, therapy or laboratory abnormality that might confound the results of the study, interfere with the patient's participation, or is not in the best interest of the patient to participate in the opinion of the Investigator including but not limited to:

Patients with history of a cerebral vascular event (stroke or transient ischemic attack), unstable angina, myocardial infarction, congestive heart failure or symptoms of New York Heart Association Class III-IV (see Appendix D) documented within 6 months prior to first dose of BT8009 or:

a. Mean resting corrected QT interval (QTcF) >470 msec
b. Any factors that increase the risk of QTc prolongation or risk of arrhythmic events such as heart failure, hypokalemia, congenital long QT syndrome, family history of long QT syndrome or unexplained sudden death under 40 years-of-age, or any concomitant medication known to prolong the QT interval
c. Any clinically important abnormalities (as assessed by the Investigator) in rhythm, conduction, or morphology of resting electrocardiograms (ECGs), e.g., complete left bundle branch block, third degree heart block
15. Known human immunodeficiency virus (HIV) or acquired immune deficiency syndrome (AIDS)
16. Patients with a positive hepatitis B surface antigen and/or anti-hepatitis B core antibody. Patients with a negative polymerase chain reaction (PCR) assay are permitted with appropriate antiviral therapy
17. Active hepatitis C infection with positive viral load if hepatitis C virus (HCV) antibody positive (if antibody is negative then viral load not applicable). Patients who have been treated for hepatitis C infection can be included if they have documented sustained virologic response of ≥12 weeks.
18. History of another malignancy within 3 years before the first dose of BT8009, or any evidence of residual disease from a previously diagnosed malignancy (excluding adequately treated with curative intent basal cell carcinoma, squamous cell of the skin, cervical intraepithelial neoplasia/cervical carcinoma in situ or melanoma in situ or ductal carcinoma in situ of the breast).
19. Systemic IV anti-infective treatment, or fever within the last 14 days prior to first dose of BT8009.
Suspicion of relevant and recent systemic viral syndrome or need for quarantine/isolation that is not resolved in the opinion of the investigator
21. Psychological, familial, sociological, or geographical conditions that do not permit compliance with the protocol and/or follow-up procedures outlined in the protocol.

Additional Exclusion Criteria Part A-2 and B-2 Nivolumab Combination Cohorts

22. Prior intolerance to immune checkpoint inhibitor
23. Known hypersensitivity to checkpoint inhibitor therapy
24. Prior organ transplant (including allogeneic)
25. Diagnosis of clinically relevant immunodeficiency
26. Active systemic infection requiring therapy
27. More than 10 mg daily prednisone equivalent or other strong immunosuppressant
28. History of autoimmune disease except type I DM well contained on insulin, alopecia or vitiligo

### History of Interstitial Lung Disease

Correlative Testing:

All patients will be required to provide archive tumor material or fresh
tumor biopsy for assessment of expression levels of Nectin-4 and additional molecular genetic characterization (i.e. assessment of specific somatic mutations, etc.). This material should be provided as a tissue block or 10-15 paraffin-dipped unstained slides.
Pre- and post-dose tumor biopsies will be collected to investigate intratumoral PK/Pharmacodynamic effects of BT8009. Pre- and one post-dose tumor biopsy will be optional except for the mandatory subset of patients in Part B (6 per cohort). The post-dose biopsy will be required in Cycle 1 after any dose as long as it is within 4 to 36 hours after the BT8009 dose. Optional biopsies for any patient may be taken in
Cycles 2, 3, or 4. Refer to the schedule of assessments (SOA) for further details.
Pre- and post-dose blood samples will also be collected to assess pharmacodynamic, response, and treatment resistance biomarkers, such as somatic mutations in circulating tumor DNA (ctDNA), ADA and pharmacogenomic analysis.

Statistical Methodology

Dose-escalation (applies separately to A-1 and A-2): The actual number of dose levels to be explored in this study will depend on determination of the non-tolerable dose based on dose-limiting toxicities (DLTs). The MTD will be defined based on DLTs (see Section 5). Other safety data, as well as PK profiles observed during the conduct of the study and any trends for anti-tumor activity. Treatment cycles will occur consecutively as per the SOA. If one patient experiences a DLT at a given dose level then an additional 3 patients will be treated with the same dose. Evaluation of a cohort of at least 3 patients completing 1 cycle of treatment (28 days) is required prior to proceeding to the next dose level. Additional details are found in Section 5.
Following evidence of tolerability in the single subject and 3+3 escalations (dose levels 1-4), all subsequent dose interval escalations will be based on a continual reassessment method (CRM) using a two-parameter Bayesian logistic regression model (BLRM) with overdose control (EWOC) principle such that the next dose level will include the highest posterior probability of a DLT occurring in the target interval (20%, 33%) among doses fulfilling the overdose criterion that there exist <25% likelihood of the dose level being found unsafe (DLT rate ≥33%). The BLRM will be applied to cumulative DLT/safety data with results being made available to the SRC to formally recommend the precise dose escalation at each dose level. An estimated escalation scheme is provided in Section 5, with full details in Appendix G: Details and Operating Characteristics of the Dose Escalation Design. See Section 5.
Futility analyses will be performed on each expansion cohort in Part B (B-1 and B-2). Simon two-stage design to test the null hypothesis that P<0.17 versus the alternative that P≥0.380 After testing the drug on the first 14 efficacy evaluable patients, the trial will be terminated if 3 or fewer respond (ORR ≥21%). If the trial goes on to the second stage, a total of 40 (26 additional patients) efficacy evaluable patients will be studied. If the total number responding is <10, then the drug will be stopped.
Therefore, the maximum number of patients recruited into the study is 146; 34 from Part A-1, 20 from Part A-2, 40 from each of the two Part B cohorts and 12 from the Part C renal cohort.

### Example 2. BT8009 Formulation

The BT8009 drug product is formulated as a sterile lyophilized powder for solution. The medicinal product is contained in a 10 mL Type I clear glass vial with a chlorobutyl stopper and aluminum seal. The labelled strength of each vial includes 21.2 mg/vial for reconstitution with 5.0 mL of water for injection (WFI). With a displacement volume of 0.3 mL, a 4 mg/mL BT8009 solution is generated (the reconstituted drug substance contains histidine, sucrose, and Polysorbate 20), and 5.0 mL of the reconstituted solution will be withdrawn to provide a 20 mg dose for further dilution with 0.9% saline and administration via IV infusion.

Description and Composition of the Drug Product

BT8009 drug product will be supplied for clinical use as a white to off white sterile lyophilised powder for reconstitution in a 10 mL Type I clear glass vial with a butyl stopper and aluminium seal. During manufacture, each vial is filled with 5.3 mL of 4 mg/mL bulk solution prior to lyophilisation, providing 21.2 mg of BT8009 per vial. At the point of use, BT8009 drug product is reconstituted with 5.0 mL of sterile WFI (to return a reconstituted volume of 5.3 mL solution) providing BT8009 at a target concentration of 4 mg/mL for further dilution with 5% dextrose prior to IV administration (infusion). The 0.3 mL excess ensures an extractable volume of 5 mL from the vial providing up to a 20 mg dose for further dilution.

Hydrochloric acid is used to adjust the pH of the fill solution to target pH 7.0 (not 7.4), within the effective buffering range of the histidine buffer. The absolute amount required is dependent of the actual amount of L-histidine employed in the batch, this is indicated as ‘quantity sufficient’ to reach pH 7.0.

The complete statement of the composition and quantitative composition of BT8009 drug product (21.2 mg/vial) is presented in Table 1.

TABLE 1
Composition of BT8009 Drug Product

| | | Concentration | Reference |
|---|---|---|---|
| Component | Function | (mg/mL) | to Standard |
| BT8009 | Drug substance | 4.0^{a} | HSE |
| L-Histidine | Buffer | 5.24 | EP, USP |
| Sucrose | Stabiliser, | 60.0 | EP, NF |
| | cryoprotectant | | |
| Polysorbate 20 | Non-ionic | 0.2 | EP/NF |
| | surfactant | | |
| Hydrochloric Acid | pH adjustment | q.s^{b} | EP |
| WFI^{c} | Solvent | q.s^{b}. to 1 mL | EP/USP |

Based on API potency, adjustments will be made to achieve the target drug substance label claim value.
^{b}Quantity sufficient
^{c}Water for injections (WFI) is driven off during lyophilisation process

1. A pharmaceutical composition comprising BT8009, or a pharmaceutically acceptable salt thereof, and a pharmaceutically acceptable excipient or carrier selected from a buffering agent, a stabilizer or cryoprotectant, and a surfactant.

2. The pharmaceutical composition of claim 1, wherein the buffering agent is histidine.

3. The pharmaceutical composition of claim 1 or 2, wherein the stabilizer or cryoprotectant is sucrose.

4. The pharmaceutical composition of any one of claims 1-3, wherein the surfactant is Polysorbate 20.

5. The pharmaceutical composition of any one of claims 1-4, further comprising a pH adjusting agent.

6. The pharmaceutical composition of claim 5, wherein the pH adjusting agent comprises hydrochloric acid.

7. The pharmaceutical composition of any one of claims 1-6, which is a solid pharmaceutical composition in lyophilized powder form.

8. The pharmaceutical composition of any one of claims 1-6, which is a liquid pharmaceutical composition further comprising water.

9. The pharmaceutical composition of any one of claims 1-8, comprising:
BT8009, or a pharmaceutically acceptable salt thereof;
about 1.31-2.62 mg histidine per mg of BT8009, or a pharmaceutically acceptable thereof;
about 15-30 mg sucrose per mg of BT8009, or a pharmaceutically acceptable thereof; and
about 0.05-0.1 mg Polysorbate 20 per mg of BT8009, or a pharmaceutically acceptable thereof.

10. The pharmaceutical composition of claim 9, comprising:
about 21.2 mg BT8009, or a pharmaceutically acceptable salt thereof;
about 27.8 mg histidine;
about 318 mg sucrose; and
about 1.06 mg Polysorbate 20.

11. The pharmaceutical composition of claim 8, comprising:
about 2-4 mg/mL BT8009, or a pharmaceutically acceptable salt thereof;
about 5.24 mg/mL histidine;
about 60 mg/mL sucrose; and
about 0.2 mg/mL Polysorbate 20.

12. A method for treating an advanced solid tumor malignancy associated with Nectin-4-expression in a patient comprising intravenously administering to the patient the pharmaceutical composition of any one of claims 1-11.

13. The method of claim 12, wherein the advanced solid tumor malignancy associated with Nectin-4-expression is selected from the group consisting of non-small-cell lung cancer (NSCLC), ovarian cancer, triple-negative breast cancer (TNBC), gastric/upper gastrointestinal (GI) cancer, pancreatic cancer, and urothelial cancer.

14. The method of claim 12 or 13, wherein the pharmaceutical composition is administered once every 7 days.

15. The method of any one of claims 12-14, wherein the pharmaceutical composition is administered at a dose of about 2.5, 5.0, 7.5, 10.0, 13.0 or 17.0 mg/m².

16. The method of any one of claims 12-15, wherein the pharmaceutical composition is administered via an IV infusion of about 60 minutes.

17. The method of any one of claims 12-15, further comprising administering Nivolumab.

18. The method of claim 17, wherein the Nivolumab is administered via an IV infusion of about 30 minutes.


SEQUENCE LISTING (1 sequence)

SEQ ID NO: 1 - protein, 17 aa, Artificial
  Synthetic Peptide
  Xaa (1)..(1): Xaa is B-Ala
  Xaa (2)..(2): Xaa is Sar10
  Xaa (5)..(5): Xaa is 1Nal
  Xaa (9)..(9): Xaa is HArg
  Xaa (15)..(15): Xaa is HyP
XXCPXDCMXDWSTPXWC